(19) United States
(12) Patent Application Publication
(10) Pub. No.: US 2026/0188436 A1
(43) Pub. Date: Jul. 2, 2026

(54) COMPUTER-IMPLEMENTED METHOD FOR MINERAL QUANTIFICATION BY X-RAY DIFFRACTION AND COMPUTER-READABLE NON-TRANSIENT STORAGE MEDIUM

(71) Applicants: PETRÓLEO BRASILEIRO S.A. - PETROBRAS, Rio de Janeiro (BR); OPTIMATECH LTDA, Rio de Janeiro (BR)

(72) Inventors: THIAGO DE SÁ FEITAL, Rio de Janeiro (BR); MAURÍCIO MELO CÂMARA, Rio de Janeiro (BR); JULIÁN ANDRÉS GARCÍA CÁRDENAS, Rio de Janeiro (BR); CAMILA WENSE RAMNANI, Rio de Janeiro (BR); GABRIEL SALVADOR CARDOSO, Rio de Janeiro (BR); PEDRO HENRIQUE DE LIMA RIPPER MOREIRA, Rio de Janeiro (BR); JOSÉ CARLOS COSTA DA SILVA PINTO, Rio de Janeiro (BR); GABRIEL DO NASCIMENTO FREITAS, Rio de Janeiro (BR); MURILO SILVEIRA GOMES, Rio de Janeiro (BR); FABIANO GALDINO LEAL, Rio de Janeiro (BR); RENATA ALVES MOTA, Rio de Janeiro (BR)

(73) Assignees: PETRÓLEO BRASILEIRO S.A. - PETROBRAS, Rio de Janeiro, RJ (BR); OPTIMATECH LTDA, Rio de Janeiro (BR)

(21) Appl. No.: 19/428,463

(22) Filed: Dec. 22, 2025

(30) Foreign Application Priority Data
Dec. 30, 2024 (BR) 1020240275241

(51) Int. Cl. G16C 20/20; G01N 23/2055; G16C 20/70

(52) U.S. Cl. CPC G16C 20/20; G01N 23/2055; G16C 20/70

(57) ABSTRACT

The present invention solves the technical and operational challenges faced by the X-ray diffractometry analysis process in the oil and gas industry through calculations that replace the manual visual steps performed by laboratory analysts. The computer-implemented method unifies all manual steps of the XRD analysis process in a single environment, also eliminating the need for multiple software programs and repetitive activities. Furthermore, the computer-implemented method uses computational search algorithms and physical constraints of the diffractometer to robustly identify minerals, significantly reducing analysis time per sample and increasing the standardization of results. This innovative and effective approach offers a complete solution to the challenges faced by the XRD analysis process in the oil and gas industry, ensuring faster, more accurate, and more efficient sample analysis.

### CROSS-REFERENCE TO RELATED APPLICATIONS

This application claims priority to Brazilian Application No. BR 1020240275241, filed Dec. 30, 2024, the disclosure of which is herein incorporated by reference in its entirety.

### FIELD

The present invention falls within the field of petroleum engineering. More specifically, the present invention relates to a computer-implemented method for mineral quantification by X-ray diffractometry and a computer-readable non-transient storage medium.

### BACKGROUND

X-ray diffractometry is a well-established technique for studying the mineral composition of rocks. In the oil and gas industry, this technique is used both during well drilling, to provide real-time information, and in subsequent studies on rock samples collected during drilling, such as sidewall samples and core samples.

The X-ray diffractometry (XRD) analysis process in the oil and gas industry is characterized by a number of technical and operational challenges. Analysts face significant difficulties, such as the fragmentation of the process across multiple software programs, the need to perform manual, repetitive tasks subject to analyst subjectivity, the lack of integration between the systems used, and the delay in obtaining and interpreting the results. These problems directly impacted the efficiency, accuracy, and reliability of the analyses performed, consequently affecting the quality of decisions made based on this information.

Due to the high demand for X-ray diffractometry analyses, it is necessary to always seek ways to improve the efficiency in obtaining this data, without losing the high accuracy of the results provided. For a good interpretation of diffractograms of rock samples, there are two fundamental steps, which are the qualification and quantification of the mineral phases present in the generated spectrum. These two steps are currently performed in different software programs, which consume time of the analysts to save files with different extensions and often convert them to be opened in another software. The qualification stage is performed by visual analysis, in which the existing mineral patterns in a database are compared with the spectrum generated in the experiment. When, visually, it is noted that the mineral tokens correspond to the generated spectrum, the quantification stage begins. In quantification, the mineral tokens selected in the qualification phase are used for refinement by the Rietveld method.

This fragmented approach results in a series of problems, such as the lack of integration between the systems used, the need to perform manual and repetitive activities, the delay in obtaining and interpreting the results, and the propensity for errors. These challenges made the XRD analysis process slow, complex, and prone to failures, compromising the efficiency and reliability of the analyses performed.

Therefore, the object of the present invention is to offer a comprehensive solution to such challenges, promoting the optimization and integration of the XRD analysis process in the oil and gas industry.

### STATE OF THE ART

The document U.S. Pat. No. 7,184,517 B2, entitled “Analytical method for determination of crystallographic phases of a sample”, discloses an analytical method for determining crystallographic phases of a measurement sample comprising the steps of acquiring a diffraction pattern of the measurement sample and qualitative phase analysis of the measured diffraction pattern, acquiring an element spectrum of the measurement sample and determining concentrations of chemical elements in the measurement sample from the acquired element spectrum, and performing a quantitative phase analysis of the measurement sample based on the measured intensities of the acquired diffraction pattern, taking into account determined element concentrations as a boundary condition, wherein the differences between calculated and measured intensities of the diffraction pattern and between calculated and determined element concentrations are simultaneously minimized in an iterative process. The method allows quantitative phase determination. The document is silent regarding the type of sample analyzed, not disclosing whether samples originating from the drilling area of oil and gas wells were tested.

The document US20240142394 A1, entitled “Crystal structure database-based material analysis method and system, and application”, discloses a material analysis method based on a crystal structure database, a system, a computer-readable storage medium, and an application. The material analysis method includes comparing experimental standard information obtained from the examination of sample to be tested with theoretical standard information calculated from material structure data in the crystal structure database and obtaining crystallographic and phase composition information of the sample to be tested through intelligent analysis. The crystallographic information includes space group, unit cell parameter, and specific coordinates of atoms in the unit cell. The crystal structure database has material structure data obtained by experimental measurement and/or theoretical prediction, including chemical formula, space group, unit cell parameter, and specific coordinates of atoms in the unit cell. The document is silent regarding the type of sample analyzed, not disclosing whether samples originating from the area of oil and gas well drilling were tested.

The document WO 2016205894 A1, entitled “Method for determining the mineral composition of a geological sample”, discloses a computer-implemented method for determining the composition of a geological sample comprising at least one mineral species by means of a controller, in which an X-ray beam is diffracted by the geological sample and captured by a detector to produce an X-ray diffraction pattern of the sample, the controller comprising a processor and a memory that stores program instructions that, when executed by the processor, cause the implementation of the following steps: (a) comparing the X-ray diffraction pattern of the sample to a plurality of X-ray diffraction patterns, each associated with at least one mineral species provided in a customized reference dataset of mineral species; (b) rejecting any obviously incompatible mineral species from the custom reference dataset to leave a remainder of potentially matching mineral species; (c) performing an optimization analysis of the X-ray diffraction pattern of the sample in relation to potentially matching mineral species to calculate the percentage of each mineral species in the sample; (d) rejecting any calculated mineral species as having a percentage below a predetermined limit; (e) repeating steps (c) and (d) until no other mineral species are rejected; and (f) generating a result comprising the calculated percentages of each mineral species in the geological sample. The document is also silent regarding the type of sample analyzed, not disclosing whether samples from the oil and gas well drilling area were tested.

### SUMMARY

The present invention consists of a computer-implemented method for mineralogical characterization of rock samples based on conventionally obtained XRD data. The computer-implemented method features a data processing step, a phase identification step, and a phase quantification step. The invention performs mathematical operations to replace the visual analysis of a human analyst, thus eliminating analyst subjectivity and minimizing errors, as well as performing the operations much faster and with greater precision.

Furthermore, the present invention relates to a computer-readable non-transient storage medium comprising instructions stored therein, wherein the instructions, when read by a computer, cause the computer to execute the steps of the method as defined above.

### BRIEF DESCRIPTION OF THE DRAWINGS

The present invention will now be described with reference to typical embodiments thereof and also with reference to the appended drawings.

FIG. 1 is a representation of a graph constructed as a result obtained by the reading and conversion step of the experimental XRD according to the present invention.

FIG. 2 is a graph showing the baseline and peaks extracted for an XRD according to the present invention.

FIG. 3 is a flowchart representing the steps and substeps of the computer-implemented method according to the present invention in operation.

FIG. 4 is a representation of the steps of the algorithm involved in the estimation of scale factors based on peak alignment, according to the present invention.

FIG. 5 is a representation of the results obtained by the method of the present invention showing an experimental diffractogram.

### DETAILED DESCRIPTION

Specific embodiments of the present disclosure are described below. In an effort to provide a concise description of these embodiments, all features of an actual implementation may not be described in the specification. It should be appreciated that in the development of any actual implementation, as in any engineering or design project, numerous specific implementation decisions must be made to achieve the specific objectives of the developers, such as compliance with system and business-related constraints, which may vary from one implementation to another. Furthermore, it should be appreciated that such a development effort may be complex and time-consuming but would nevertheless be a routine design and manufacturing undertaking for those of common ability having the benefit of this disclosure.

The present invention describes a computer-implemented method for mineral quantification by X-ray diffractometry. The method can be used in sectors that employ X-ray diffractometry analysis, such as the chemical, mining, and materials industries. Notably, the invention has wide application in the oil and gas industry, facilitating the mineralogical characterization of rock samples from production fields. X-ray diffractometry is a well-established technique for studying the mineral composition of rocks, being used both during well drilling, to provide real-time information, and in subsequent studies on rock samples collected during drilling, such as sidewall samples and core samples. The application of the computer-implemented method allows for faster, more accurate, and efficient analysis of samples, contributing to safer and more strategic decision-making regarding oil exploration and production.

Mineral quantification by X-ray diffractometry (XRD) analysis begins with sample preparation and subsequent acquisition of experimental scattering data produced when an X-ray beam interacts with the sample, where the diffracted X-rays are detected by a detector, which measures the light intensity as a function of the scattering angle (2θ). The equipment used in this analysis exports the experimental data obtained in binary files with the .raw extension, the format of which may vary depending on the XRD instrument and the software used for data collection. Thus, there are several different file formats, although using the same .raw extension, which depend on the manufacturer of the equipment used (e.g., Rigaku and Bruker). Therefore, it is necessary to perform procedures for loading the XRD data.

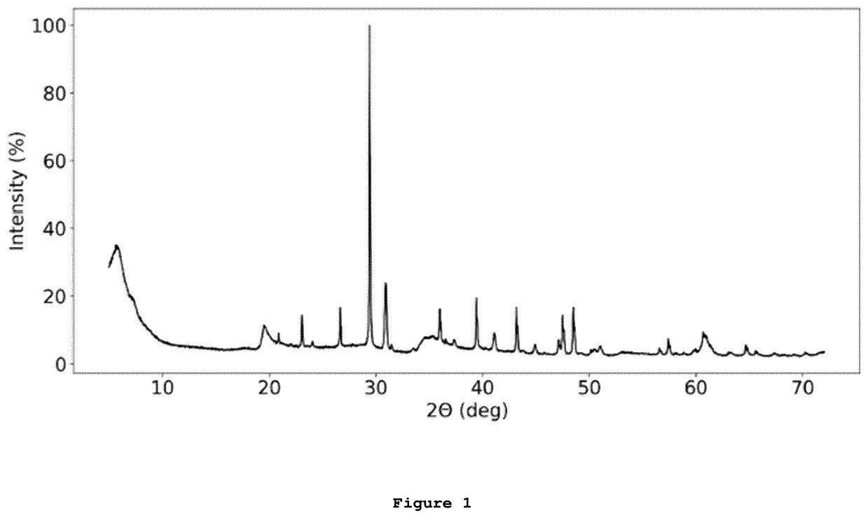

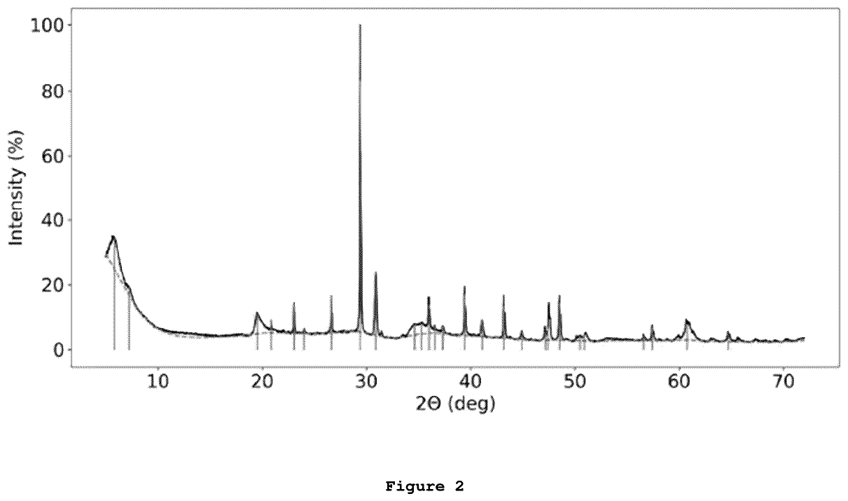

FIG. 1 shows an example of a graph constructed as a result obtained by the reading and conversion step of the experimental XRD. FIG. 2 shows an example of a graph showing the baseline and peaks extracted for an experimental XRD, where the black line represents the experimental diffractogram, the red dashed line represents the baseline, and the blue lines represent the extracted peaks. Both are obtained by means already known from the state of the art.

In the known technique, the peaks of the experimental diffractogram are identified by comparison with peak information for reference samples with known composition, or mineral standard sheets, also called phases. The peak information of the phases is stored and distributed in the .CIF (Crystallographic Information File) file format, a standard file format developed specifically for the exchange of crystal structure data. .CIF files cannot be used directly and must be converted to a file format with the extension .str. This conversion can be performed with software such as Profex (https://www.profexxrd.org/).

At this point, the previous technique proceeds to visual comparison between the peaks of the sample and peaks of a phase bank, performed by a human. As mentioned earlier, this process is time-consuming and subject to individual subjectivity, as well as being prone to errors.

The present invention provides an objective methodology for determining the mineral composition of the tested samples in an automated manner and free from human subjectivity. Furthermore, the methodology described below can be implemented in a single application or computer program. These characteristics give the method of the present invention great speed and precision.

The method of the present invention has three stages, Treatment, Identification, and Quantification, each subdivided into several sub-stages. Initially, a set of experimental diffractogram data is obtained as known in the state of the art. The first stage of the method of the present invention, Treatment, will first treat this data so that it can be used by the following stages, all within the same computational environment and without the need for multiple conversions performed by different software.

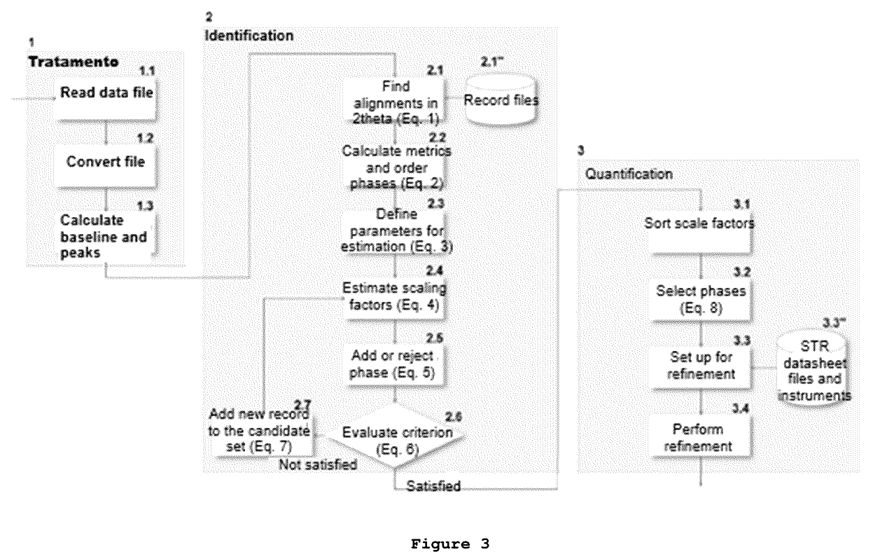

The computer-implemented method program of the present invention will now be detailed with reference to FIG. 3.

### 1—Treatment.

The method begins in the Treatment stage 1, with the receipt of experimental diffractogram data in uncompressed and unprocessed image data files, such as the .RAW format obtained by one or more x-ray diffractometers. There are several different file formats, although they use the same. RAW extension. The equipment used in this analysis exports the experimental data obtained in binary files with the raw extension, whose standard for creating the binary file may vary depending on the XRD instrument and the software used for data collection.

### 1.1—Read Data File

The received files are read by the computer where the invention is implemented.

### 1.2—Convert File

The data file needs to be converted to an accessible format, in which it is possible to extract the data and load it in matrix form. For this, a conversion of the received files to a text file in .xy format is performed, and from the .xy file, the actual loading of the experimental diffractogram data. The matrix is formed by two columns, the first being the angle 2θ and the second being the light intensity, while the rows are generated by the angular movement of the diffractometer, reaching thousands of points, forming a spectrum or diffractogram. This matrix can generate a graph between the angles 2θ on the abscissa axis and the corresponding intensities on the ordinate axis, as seen in FIG. 1. In X-ray diffraction, the dispersion angle (2θ) is related to the spacing between the diffraction planes (d) in a crystal lattice by means of the Bragg equation, d=λ/(2 sin(θ)), where A is the wavelength of the X-ray.

### 1.3—Calculate Baseline and Peaks

The data matrix obtained in 1.2 is the input argument of a method that will calculate the baseline of the spectrum; that is, a line that passes through the points with the lowest intensities and eliminates unwanted effects in the diffractogram, highlighting only sections with abrupt variations in intensity, commonly called peaks. The calculation of the baseline is done based on the baseline algorithm with asymmetric least squares smoothing (Baseline Correction with Asymmetric Least Squares Smoothing. Paul H. C. Eilers and Hans F. M. Boelens. Oct. 21, 2005). Then, the data are entered into a peak extraction method, based on the height between the diffractogram and the baseline. Peak identification is performed using a wavelet transform method (Pan Du, Warren A. Kibbe, Simon M. Lin, Improved peak detection in mass spectrum by incorporating continuous wavelet transform-based pattern matching, Bioinformatics, Volume 22, Issue 17, September 2006, Pages 2059 to 2065, https://doi.org/10. 1093/bioinformatics/bt1355). Only the data relating to the peaks of the experimental diffractogram are used in the following steps.

### 2—Identification

Next, the method moves to the Identification step 2, where the peaks of the experimental diffractogram and the reference peaks, also called tokens, contained in a database 2.1″ are compared to determine the types of minerals contained in the rock samples tested.

### 2.1—Find Alignments in 2θ

Identification of the points where alignment occurs between the sample peaks and the phase peaks, searching for the set of sample-token angle (or diameter) pairs whose difference is less than an established tolerance, following Equation 1:

$$Ak = \{ ( dj , dkm ) \} : ‖ dj - dkm ‖ \leq tol , ∀ j , k , m \qquad ( 1 )$$

where: d is the diameter, k is the token of phase, j is the sample peak, m is the peak of the record and tol is the specified tolerance. The tolerance is chosen based on the type of equipment used to perform the XRD (before step 1). The equipment (e.g., Rigaku and Bruker) has a specific precision that depends on the instrument; this precision is expressed in terms of angle and indicates the uncertainty of the intensity measurement with respect to the angle of incidence of the X-ray beam.

### 2.2—Calculate Metric and Order Phases

Based on a metric that consolidates the number of alignments found the sample and each phase (Equation 2), the probability that phase k (i.e., a token) is in the sample is calculated, followed by an ordering of the phase list in the decreasing direction of the metric, where the phases with the highest percentage of alignments are ranked at the top of the list. It is assumed that the higher the percentage of peaks found between a phase and the sample, the greater the probability that this phase is present in the sample. This is performed according to Equation 2:

$$p k = n ( Ak ) n ( j ) n ( m ) k , ∀ j , k , m \qquad ( 2 )$$

where: pk is the percentage of alignments between a diffractogram and a phase k, n(Ak) is the number of elements in the set Ak, n(j) is the number of peaks in the sample and n(m)k is the number of peaks in the k-sheet.

### 2.3—Define Parameters for Estimation

Definition of the parameters, by Equation 3, to be used for the estimation of scale factors, to be used in the phase selection step (3.2).

$$\begin{matrix}{ϵ}_{0} = 1 * {10}^{6} \\ q = 1 \\ r = \{ k \} : k := \{ {( {p}_{k} )}_{q} \}\end{matrix} \qquad ( 3 )$$

where ε₀ is the initial value of the error and high enough to start the procedure, q is the counter that indicates the ordinal in the set of probabilities and r is the set of phases identified in the sample.

### 2.4—Estimate Scale Factors

Estimation of the scale factor for a phase, which consists of finding the scaling factors of each of the k chips that best construct the sample spectrum, following the order of substep 2.2. parameter estimation is performed using the least squares criterion between the intensities of the experimental diffractograms and the scaled peaks of the phase, as seen in Equation 4:

$$\begin{matrix}ϵ = {min}_{x ϵ {ℝ}^{\# τ}} {\sum }_{j = 1}^{\# j} {( {I}_{j} - M x )}^{2} , \\ M ∈ {ℝ}^{\# j \times \# r} := \{ ( {I}_{{r}_{1} m} ) , ( {I}_{{r}_{2} m} ) , … , ( {I}_{{r}_{\# τ} m} ) \}\end{matrix} \qquad ( 4 )$$

where I is the vector of sample intensities, x∈ [symbol] n(r) is the vector of scale factors, M∈ [symbol] n(j)×n(r)={(l(r)1m,(l(r)2m), . . . , (l(r)n(r)m)} is the matrix with the intensities of the phases of the set r. The columns correspond to the intensities of each phase and each row corresponds to the intensity Ij aligned in the respective 2θ.
2.5—Add or reject phase

Evaluation of the criterion for adding the phase from the record to the set r, based on the comparison of the deviation metric (substep 2.4) with the Reference value (substep 2.3) combined with the total number of peaks (Equation 5.1), also rejecting phases with negative scale factors (Equation 5.2):

$$ϵ \geq ϵ 0 ⇒ r ← r - \{ k \} \qquad ( 5.1 )$$

$$xi \leq 0 ⇒ r ← r - \{ ( r ) i \} , ∀ i ; \qquad ( 5.2 )$$

### 2.6—Evaluate Criterion

Comparison of the number of iterations with a reference value (Equation 6). The definition of the reference value is done empirically, guaranteeing the convergence of the algorithm. If the criterion is satisfied, the method will go to the next step (Quantification 3). Otherwise, the phase is added to the set r and it returns to substep 2.4 evaluating the next phase of the ordered list of phases (Equation 7), obtained in substep 2.2.

$$n ( r ) \leq 40 \qquad ( 6 )$$

$$q ← q + 1 ; r ← r ⋃ k , k := \{ {( {p}_{k} )}_{q} \} \qquad ( 7 )$$

where K is the final set of phases for quantification.

### 3—Quantification

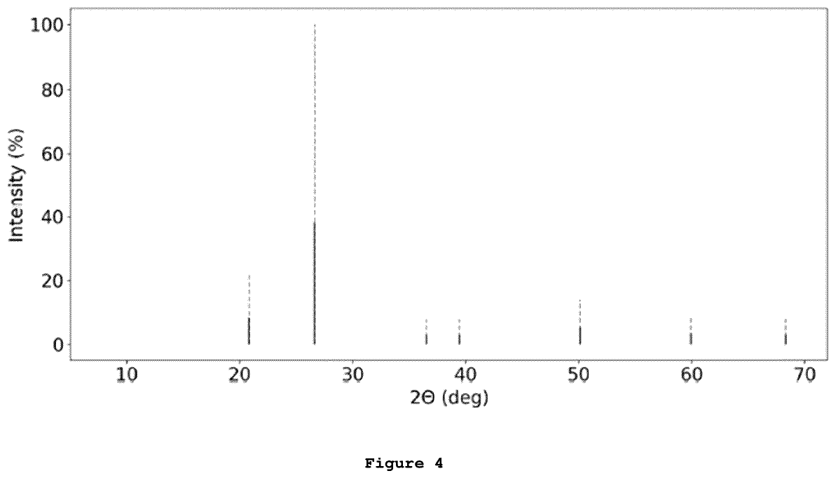

The final list of phases and their respective scaling factors are used in this step. FIG. 4 illustrates the steps of the algorithm involved in estimating the scaling factors based on the alignment of the peaks, with the peaks with the original intensity from the record of the element (dashed line, red) and the peaks with intensity adjusted using the scaling factor (solid line, black). With the minerals identified, the configuration and execution of the BGMN module is carried out, which contains an implementation of the Rietveld method, a technique used to refine the structure and parameters of a crystalline material from its x-ray diffraction pattern through a fundamental approach (based on x-ray diffraction phenomena) from the quantification of the phases and refinement of the structures, allowing the reconstruction of the diffractogram. BGMN software is a data analysis software that implements the Rietveld technique (http://www.bgmn.de/). Another relevant software that implements the Rietveld technique is GSAS-II (https://advancedphotonsource.github.io/GSAS-II-tutorials/).

### 3.1—Order Scale Factors

At the end of the last iteration of the parameter estimation problems, the scale factors for all phases are obtained, which can represent (to a certain extent) a quantity measure for each phase. Thus, the phases are ordered according to the corresponding scale factor using Equation 8.

$$K = \{ k \} , k := \{ ( {x}_{k} ) \} , k = 1 , 2 , … , 15 \qquad ( 8 )$$

where K is the final set of phases for quantification.

### 3.2—Select Phases

Selection of phases with the largest scale factors through successive estimations (Equation 9), and evaluation of alignment in I (Equation 10). The preferred number of selected phases was experimentally determined to be 15, however, the method is not restricted to this number. Particular applications may use a different number of phases with the largest scale factors.

$$x 1 \to x 1 , x 2 \to x 1 , x 2 , x 3 \to … \qquad ( 9 )$$

$$e 1 = ( I - I 1 \times 1 ) \to e 2 = ( I - I 1 \times 1 - I 2 \times 2 ) \to … \qquad ( 10 )$$

where the acceptance criteria are x1>0, and ek−ek−1<0, given a number n of iterations, for example, 40 iterations.

### 3.3—Configure for Refinement

With the experimental diffractogram, the selected phases and their corresponding 3.3″ sheets (files in .STR format), preparation is made for performing the Rietveld method, which is a parameter estimation, commonly called refinement.

### 3.4—Perform Refinement

Execution of the Rietveld method from the BGMN module to determine the quantities of each phase present in the sample. The data contained in a phase sheet are used to generate a calculated diffractogram, which is compared with the experimental diffractogram in a weighted least squares function. Some of these data contained in the sheets are refined (slightly modified) so that the fit is improved. Thus, a corresponding quantity is obtained for each phase present in the rock sample tested.

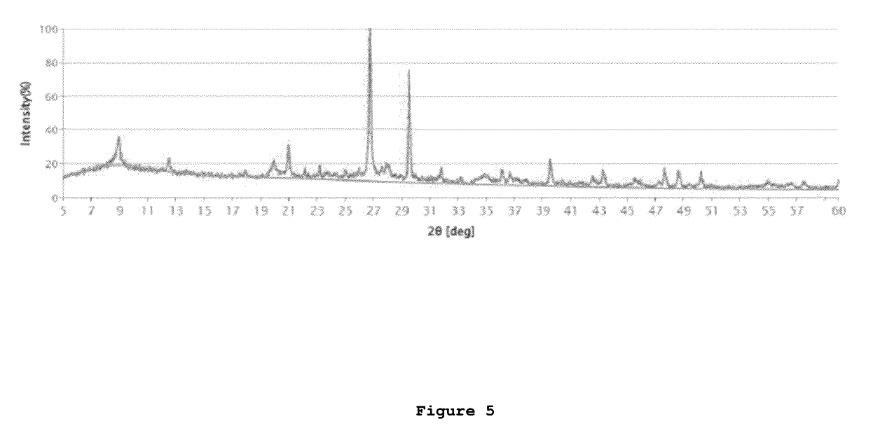

FIG. 5 shows an example of an experimental diffractogram (solid blue line) and a calculated diffractogram (dashed yellow line) along with the baseline (purple line), obtained with quantification by the Rietveld method using the BGMN after execution of the proposed method, for qualitative evaluation of the performance of the Rietveld method refinement.

Optionally, an additional step 3.5 can be performed to indicate the results in the form of graphs and tables, to provide the necessary information to the analysts and geologists involved in this process in a clear and concise manner. An example is provided in Table 1 below.

TABLE 1

| Groups/ | | |
|---|---|---|
| Minerals | Description | % by mass |
| A + M | Clay minerals + | 43.01 |
| | phyllosilicates | |
| QTZ | Quartz | 20.15 |
| CAL | Calcite | 17.84 |
| PLG | Plagioclase | 9.89 |
| KFD | K-Feldspar | 3.32 |
| PIR | Pyrite | 1.88 |
| BAR | Barite | 1.83 |
| PRX | Pyroxene | 1.37 |
| DOL - ? | Dolomite | 0.42 |
| GTH - ? | Goethite | 0.3 |

The advantages of the present invention in relation to the state of the art will become evident to those skilled in the art and include:
Simplification of the mineralogical characterization process, from the reduction in the number of software programs used in the activity;
Reduction of cost associated with software licenses, necessary for characterization in the current methodology;
Increased speed of mineralogical characterization, increasing laboratory productivity, since the time with the method of the present invention is 5 minutes, and the time with the state of the art is 30 minutes. In laboratory analyses and, also, in drilling, the time reduction is significant, in addition, the interpretation has accuracy equal to or greater than the prior art;
Increased capacity to perform mineralogical characterization analyses for the same installed capacity and the same level of human resources, from the increase in productivity;
Reduction of the unit cost to 16% of the value of the mineralogical characterization analysis of the state of the art, based on increases in productivity and execution capacity, while maintaining accuracy equal to or greater than the state of the art;
Standardization of mineralogical characterization analyses with objective and quantifiable parameters, reducing the level of subjectivity inserted by the analyst in the identification and quantification of phases;
Enablement for the creation and development of new search and modeling functions, especially by artificial intelligence methods, based on the construction of an integrated system with database resources.

The results achieved with the implementation of the computer-implemented method described herein will be evident to those skilled in the art. The integration and optimization of the XRD analysis process provided by this method result in a substantial reduction in analysis time per sample, corresponding to an average reduction of 84% (as per data in Table 2), a reduction in operating costs from the replacement of multiple commercial software programs with a single system, elimination of the need for manual and repetitive activities, increased standardization of results, and significant simplification of the work of the analysts.

The method implemented by computer program is also applied in the process of drilling new oil wells, performing analyses of the extracted rocks and enabling corrections and adjustments to the process and equipment in real time, whether in an offshore or onshore environment. This allows for a significant reduction in oil exploration costs, since the mineralogy results indicate the presence and quantity of minerals that can harm drilling, damaging the drill bit or the well structure. Thus, with the increase in mineralogical information throughout the drilling process, the risk of damage to equipment is substantially reduced, as well as the cost of repairs and replacement parts, the time of unscheduled downtime, and the total cost of renting drilling assets. These results not only improve operational efficiency but also contribute to safer, more reliable, and strategic decision-making regarding oil exploration and production.

### Example of Implementation

Table 2 below shows the performance of the proposed method for an outcrop dataset from the Salta Basin, highlighting the high efficiency of the methodology in relation to the procedure from the state of the art, conducted by an analyst, highlighting an average execution time of 74 seconds for a list of 273 samples, while the procedure from the state of the art obtained an average time between 30 and 60 minutes per sample.

TABLE 2

| | | | Analysis |
|---|---|---|---|
| | | Analysis | duration - |
| | | duration - | present |
| Internal | Number of | state of the | invention |
| sample code | Samples | art (hh:mm:ss) | (hh:mm:ss) |
| 08_2014 | 160 | 01:41:36 | 00:00:39 |
| 41_19 | 28 | 00:30:49 | 00:01:06 |
| 63_17 | 25 | 00:33:08 | 00:01:19 |
| 72_14 | 17 | 00:24:37 | 00:01:28 |
| 21_12 | 9 | 00:07:55 | 00:00:53 |
| 08_17 | 5 | 00:06:42 | 00:01:24 |
| 00_00 - mixed | 10 | 00:18:21 | 00:01:51 |
| 25_21 | 2 | 00:03:03 | 00:01:30 |
| 21_19 | 4 | 00:05:27 | 00:01:15 |
| 82_17 | 6 | 00:05:33 | 00:00:55 |
| 95_15 | 7 | 00:09:22 | 00:01:19 |
| Total | 273 | 04:06:33 | 00:13:38 |

Finally, in another embodiment of the present invention, a computer-readable non-transient medium is provided. The medium may be, for example, a memory, a flash memory, a hard disk, a compact disk, or any other device capable of storing computer instructions. When the readable medium of the present embodiment is read by a computer, the computer is enabled to perform the mineral quantification method by X-ray diffractometry as disclosed above.

Although aspects of the present disclosure may be susceptible to several modifications and alternative forms, specific embodiments have been shown by way of example in the drawings and have been described in detail in this document. But it should be understood that the invention is not intended to be limited to the particular forms disclosed. Instead, the invention should cover all modifications, equivalents and alternatives that fall within the scope of the invention, as defined by the following appended claims.

What is claimed is:

1. A computer-implemented method comprising:
(a) receiving an uncompressed and unprocessed experimental diffractogram data file obtained by one or more X-ray diffractometers;
(b) reading the data file;
(c) converting the file from a .raw format to a .xy format to obtain a data matrix;
(d) calculating baseline and peaks;
(e) finding alignments in 2θ following Equation 1:
$$Ak = \{ ( dj , dkm ) \} : ‖ dj - dkm ‖ \leq tol , ∀ j , k , m \qquad ( 1 )$$
wherein d is diameter, k is the phase token, j is sample peak, m is token peak and tol is the specified tolerance;
(f) calculating metric and order phases according to Equation 2:
$$p k = n ( Ak ) n ( j ) n ( m ) k , ∀ j , k , m \qquad ( 2 )$$
wherein pk is the percentage of alignments between a diffractogram and a phase k, n(Ak) is the number of elements in the set Ak, n(j) is the number of peaks in the sample and n(m)k is the number of peaks in the k-sheet;
(g) defining parameters for estimation according to Equation 3:
$$\begin{matrix}{ϵ}_{0} = 1 * {10}^{6} \\ q = 1 \\ r = \{ k \} : k := \{ {( {p}_{k} )}_{q} \}\end{matrix} \qquad ( 3 )$$
wherein ε₀ is the initial value of the error, q is the counter that indicates the ordinal in the set of probabilities and r is the set of phases identified in the sample;
(h) estimating scaling factors according to Equation 4:
$$\begin{matrix}ϵ = {min}_{x ϵ {ℝ}^{\# r}} {\sum }_{j = 1}^{\# j} {( {I}_{j} - M x )}^{2} , \\ M ∈ {ℝ}^{\# j \times \# r} := \{ ( {I}_{{r}_{1} m} ) , ( {I}_{{r}_{2} m} ) , … , ( {I}_{{r}_{\# r} m} ) \}\end{matrix} \qquad ( 4 )$$
wherein I is the vector of sample intensities, x∈ [symbol] n(r) is the vector of scale factors, M∈ [symbol] n(j)×n(r)={(l(r)1m,(l(r)2m), . . . , (l(r)n(r)m)} is the matrix with the intensities of the phases of the set r, columns correspond to the intensities of each phase and each row corresponds to the intensity aligned in the respective 2θ;
(i) adding or rejecting phase (based on the comparison of the deviation metric obtained in step (h) with the reference value obtained in step (g) combined with the total number of peaks, according to Equation 5.1, and rejecting phases with negative scale factors, according to Equation 5.2:
$$ϵ \geq ϵ 0 ⇒ r ← r - \{ k \} \qquad ( 5.1 )$$
$$xi \leq 0 ⇒ r ← r - \{ ( r ) i \} , ∀ i ; \qquad ( 5.2 )$$
(j) evaluating criterion according to Equation 6:
n(r)≤40 (6)
(k) responsive to the criterion not being met:
adding the current phase to the set r and returning to step (h) to evaluate the next phase in the ordered list of phases following Equation 7:
$$q ← q + 1 ; r ← r ⋃ k , k := \{ {( {p}_{k} )}_{q} \} \qquad ( 7 )$$
wherein K is the final set of phases for quantification, and
(l) responsive to the criterion being met:
ordering scaling factors of the selected phases;
(m) selecting phases comprising selecting the 15 phases with the largest scaling factors through successive estimations, according to Equation 9, and evaluating the alignment in I, according to Equation 10:
$$x 1 \to x 1 , x 2 \to x 1 , x 2 , x 3 \to … \qquad ( 9 )$$
$$e 1 = ( I - I 1 \times 1 ) \to e 2 = ( I - I 1 \times 1 - I 2 \times 2 ) \to … \qquad ( 10 )$$
wherein the acceptance criteria are x1>0, and ek−ek−1<0, given a number n of iterations;
(n) configuring for refinement with phase in the sheets corresponding to the identified phases; and
(o) performing refinement by executing the Rietveld method for the identified chips to obtain a calculated diffractogram and comparing it with the experimental diffractogram in a weighted least squares function.

2. The computer-implemented method of claim 1, further comprising presenting results obtained in the form of graphs and tables.

3. The computer-implemented method of claim 1, wherein instructions, when read by a computer, cause the computer to execute the steps of the method.

4. The computer-implemented method of claim 2, wherein instructions, when read by a computer, cause the computer to execute the steps of the method.
